(19) United States
(12) Patent Application Publication
(10) Pub. No.: US 2025/0270238 A1
(43) Pub. Date: Aug. 28, 2025

(54) A NEW BIFUNCTIONAL LINCHPIN THAT REACTS SELECTIVELY WITH CYSTEINE RESIDUES TO INVOKE CYCLIZATION WHILE CONCURRENTLY INTRODUCING A DOTAGA MOIETY FOR RADIOMETAL CHELATION

(71) Applicant: The Governors of the University of Alberta, Edmonton (CA)

(72) Inventors: Frank WUEST, Edmonton (CA); Richard YUEN, Edmonton (CA)

(21) Appl. No.: 18/867,558

(22) PCT Filed: May 19, 2023
(86) PCT No.: PCT/CA2023/050699

§ 371 Date: Nov. 20, 2024

Related U.S. Application Data
(60) Provisional application No. 63/344,138, filed on May 20, 2022.

(51) Int. Cl. C07F 7/08; C07D 403/10; C07D 403/12

(52) U.S. Cl. CPC C07F 7/0812; C07D 403/10; C07D 403/12; C07B 2200/05

(57) ABSTRACT

The present disclosure relates generally to new bifunctional linchpins that react selectively with cysteine residues to invoke cyclization while concurrently introducing a radiometal chelation ligand or silicon-based fluoride acceptor (SiFA) motif.

### FIELD

The present disclosure relates generally to new bifunctional linchpins that react selectively with cysteine residues to invoke cyclization while concurrently introducing a radiometal chelation ligand or silicon-based fluoride acceptor (SiFA) motif.

### BACKGROUND

Peptides are commonly used as pharmaceuticals and radiopharmaceuticals. However, they often need to be modified to improve their in vivo half-life.

### SUMMARY

In one aspect, there is provided a compound of Formula Y-L-R, wherein R is

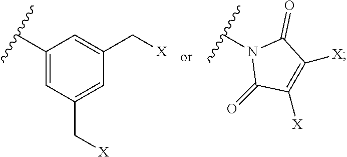

and X is a halogen, mesylate, or tosylate;
and L is an optional linker;

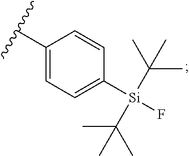 [SMILES: *c1ccc([Si](F)(C(C)(C)C)C(C)(C)C)cc1]

and Y is a radiometal chelator or
and wherein the compound is optionally further labeled with a radiometal.

In some examples, the linker may be an alkyl chain, or it may be more complex. It will be appreciated that the skilled worker would readily choose a linker to vary the length between the cyclization motif and the chelator, and could use available chemistry to do so. In one example, a commercially available radiometal chelator may have a linker extension with a free amine on the end (for example, 2-S-(4-Aminobenzyl)-1,4,7-triazacyclononane-1,4,7-triacetic acid), which may be used as a starting material in Scheme 5 and react directly with the dibromomaleimide. It will be appreciated that the chelator and linker together is still called a chelator in the art, so in some examples the linker is already built into the chelator.

In one example, the halogen is Cl, Br, or I.

In one example, the mesylate is methanesulfonyl.

In one example, the tosylate is p-methylphenylsulfonyl.

In one examples, the radiometal chelator is selected from one of:

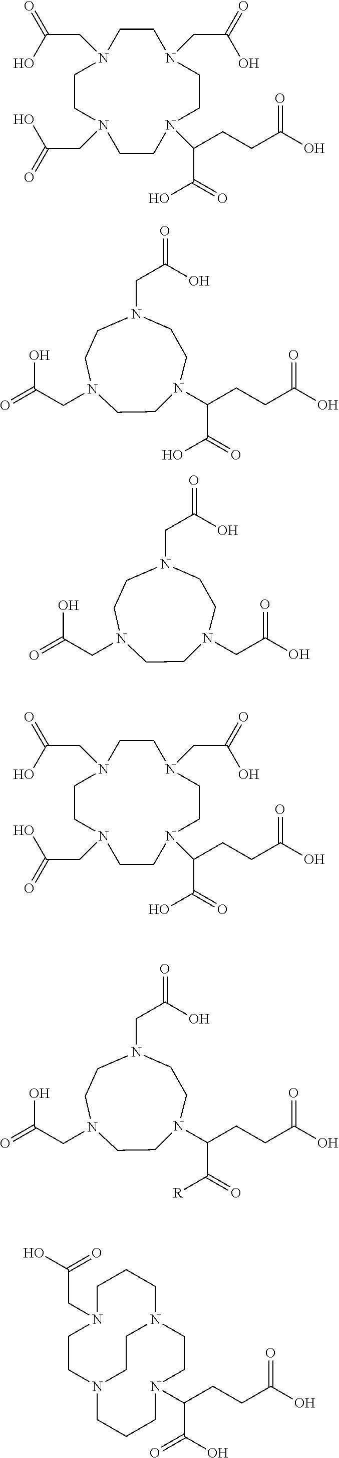

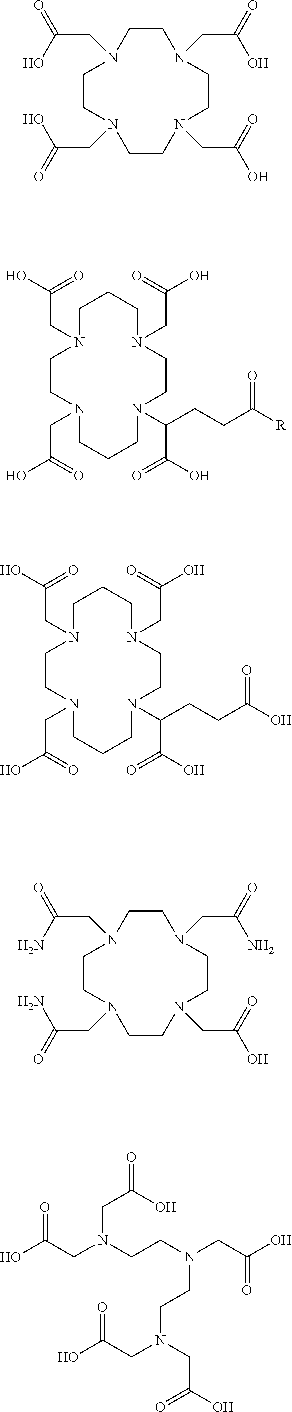

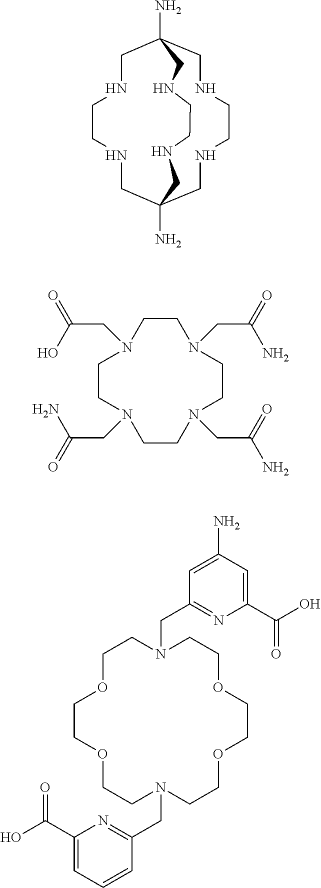

In one example, the radiometal is I¹²⁵, ⁶⁷Cu, ⁶⁸Ga, or ¹³⁵La. A skilled person may choose to incorporate any other readily available radiometal that can suitably be bound by a radiometal chelator.

### DETAILED DESCRIPTION

Peptides are commonly used as pharmaceuticals and radiopharmaceuticals. However, they often need to be modified to improve their in vivo half-life. One of the methods used for this involves peptide cyclization. In one aspect, herein we report DOTAGALP1, a new bifunctional linchpin that reacts selectively with cysteine residues to invoke cyclization while concurrently introducing a DOTAGA moiety for radiometal chelation.

Those skilled in the art will recognize that other radiometal chelating moieties and derivatives of such radiometal chelating moieties may be substituted in place of DOTAGA, including but not limited to the chelators shown in Scheme 4.

In another aspect, in addition to DOTAGALP1, a different class of bifunctional linchpins has been developed. These linchpins involve the use of a dibromomaleimide as the cyclization motif. While chelators continue to be explored, a SiFA-motif (for silicon-acceptor chemistry) can also be incorporated into the structure to access 18F-labelled compounds (SiFALP) easily.

### EXAMPLES

### Example 1

### Synthesis of DOTAGALP1

DOTAGA anhydride and 3,5-bis(hydroxymethyl)aniline, both of which are commercially available reagents, are reacted together in the presence of 1 equivalent of triethylamine (Et3N) in N,N-dimethylformamide (DMF) at 75° C. for 16 hours. The concentration of DOTAGA anhydride in DMF can vary, but usually in the range of 0.03-0.05 M. H₂O is subsequently added to quench the reaction, followed by reduced pressure evaporation to remove all of the H₂O and DMF. The crude product is purified by reverse-phase flash chromatography (using C18 silica) using a gradient of 2% CH₃CN/H₂O to 40% CH₃CN/H₂O, providing intermediate A as a white powder after lyophilization. Subsequently, A is dissolved in 33% HBr/AcOH and stirred for 16 hours at room temperature. The concentration of A in AcOH was usually in the range of 0.03-0.05 M. After completion of the reaction, diethyl ether (Et2O) is added to precipitate the DOTAGALP1, and the entire vessel was cooled in an ice bath to ensure complete precipitation. After 10 minutes of cooling, the precipitate was filtered using a fritted syringe and was washed with additional Et₂O to remove excess HBr and AcOH. The precipitate was then dissolved in deionized water and lyophilized to provide DOTAGALP1, which can be directly used to cyclize peptides without further purification.

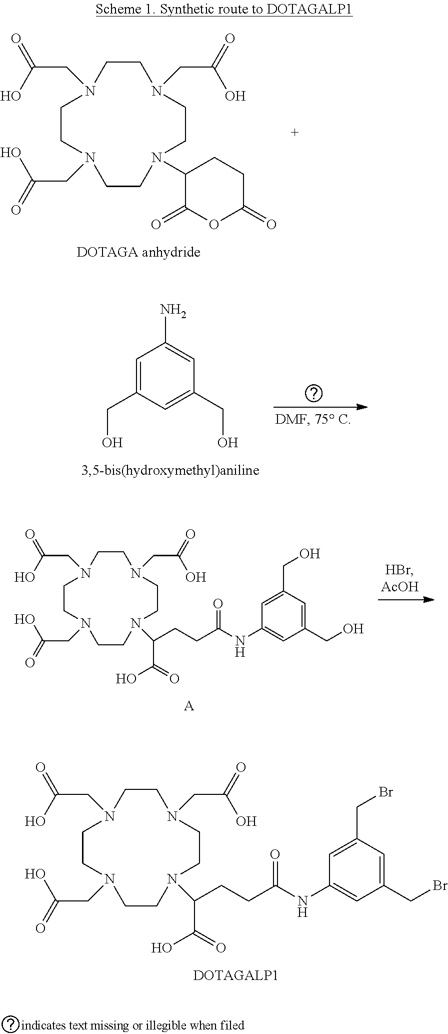

Our model peptide of choice was octreotide, which is a somatostatin receptor 2 inhibitor. Starting with octreotide acetate, the disulfide bridge was cleaved in 30 minutes using 2 equivalents of TCEP-HCl (tris(2-carboxyethyl)phosphine hydrochloride) in pH 8.5 20 mM ammonium bicarbonate buffer. Following the cleavage, a solution of 1.7 equivalents of DOTAGALP in 20 mM ammonium bicarbonate was added to get a final peptide concentration of 0.003 M. This reaction was shaken for 3 hours at room temperature. The mixture was then trapped on a primed C18 cartridge (5 mL EtOH followed by 10 mL H₂O), washed with 10 ml of water, and eluted with 3 mL of 70% CH₃CN/H₂O. The eluent was concentrated to remove most of the CH₃CN, and the cyclized peptide was purified by reverse-phase HPLC. The cyclized peptide conjugate was characterized by MALDI-TOF mass spectrometry.

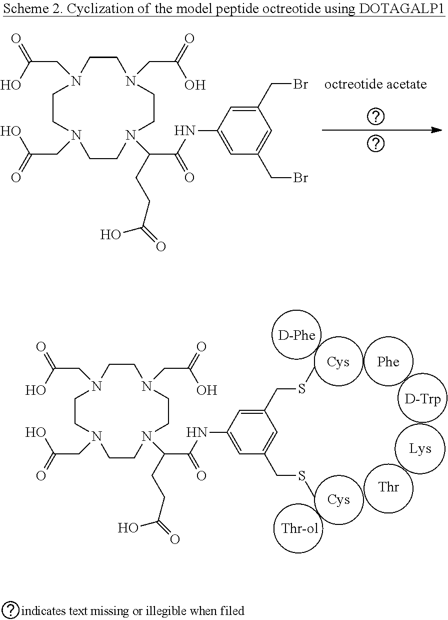

The peptide was labeled with several metals to demonstrate the wide scope of chelation possible. This included Cu, Dy, Er, Ga, Gd, In, La, Lu, Sc, and Y. The peptide was dissolved in 0.5 mL of 0.4 M NH₄OAc buffer (pH 5) followed by addition of a solution of 5-10 mg of the corresponding metal (CuCl₂, or MX₃) in 0.1 mL of 0.05 M HCl (trace metal grade). This was then reacted at 90° C. for 15 min. The chelates were then trapped on a primed C18 cartridge (5 mL EtOH followed by 10 mL 0.2% TFA in H₂O). The cartridge was washed with 8 mL of 0.2% TFA in H₂O to remove excess unreacted metal, followed by 4 mL of 70% EtOH to elute the desired compound. The chelates were also characterized using MALDI-TOF.

### IC50 Determination

As shown in Table 1, the nine octreotide derivatives were then subjected to a radioligand binding competition assay on recombinant human SSTR1-SSTR5. This was accomplished using filtration-based assays against the radiolabeled peptide ^{[125}I]SST14. The reference compound used was the peptide SST28. The results show that the octreotide derivatives are maintain selectivity for SSTR2, and that the Cu-containing chelate had the highest potency.

TABLE 1
IC₅₀ values (nM) of compounds against somatostatin
receptors (SSTR) in [¹²⁵I]SST14 binding competition

| Compound | SSTR1 | SSTR2 | SSTR3 | SSTR4 | SSTR5 |
|---|---|---|---|---|---|
| SST28 ref. | 1.32 | 0.32 | 0.35 | 2.00 | 0.10 |
| Octreo-Cu | >10000 | 110 | 2095 | >10000 | 3275 |
| Octreo-Dy | >10000 | 284 | 4450 | >10000 | 8007 |
| Octreo-Ga | >10000 | 5993 | >10000 | >10000 | >10000 |
| Octreo-Gd | >10000 | 373 | 9794 | >10000 | >10000 |
| Octreo-In | >10000 | >10000 | >10000 | >10000 | >10000 |
| Octreo-La | >10000 | 432 | 4878 | >10000 | 4650 |
| Octreo-Lu | >10000 | 1996 | >10000 | >10000 | >10000 |
| Octreo-Sc | >10000 | 233 | 5394 | >10000 | 3184 |
| Octreo-Y | >10000 | 309 | 3581 | >10000 | 6632 |

The peptide was also successfully radiolabeled with ⁶⁷Cu, ⁶⁸Ga, and ¹³⁵La using common DOTA labeling procedures. ⁶⁷Cu labelling was performed in NH₄OAc buffer pH 5.5 in 10 min at 95° C. using 1 μg of the octreotide derivative. Using 2.5-2.6 MBq in 50 μL total volume gave 96.4±0.9% decay corrected radiochemical yield (n =4). ⁶⁸Ga labelling was performed in NaOAc buffer pH 4.5 in 15 min at 90° C. using 10 μg peptide. Using 70-160 MBq in 1 mL total volume gave 90.3±2.7% d.c. RCY (n=3). ¹³⁵La labelling was performed in NaOAc buffer pH 5 in 10 min at 90° C. using μg peptide. Using 2.8-3.0 MBq in 50 μL total volume gave 83.4±6.0% d.c. RCY (n=3).

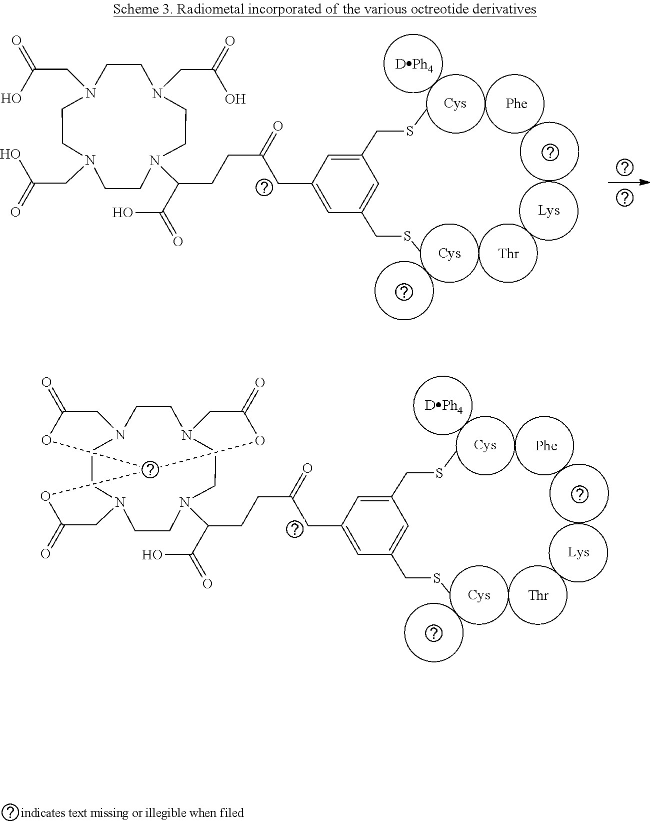

### Conclusion

DOTAGALP1 was successfully synthesized and used to cyclize octreotide. The resulting conjugates were labelled with several different non-radioactive metals and tested in vitro. The new octreotide derivatives were found to maintain specificity for SSTR2 over the other SSTR isoforms and the Cu-labelled derivative showed the best potency. While the potency was several orders of magnitude higher than the SST28 reference peptide, the IC₅₀ was still in the nanomolar range. The octreotide derivative was also successfully radiolabeled with ⁶⁷Cu, ⁶⁸Ga, and ¹³⁵La, demonstrating the broad utility of the DOTAGA moiety. While we have demonstrated a non-limiting example of DOTAGALP1 used to cyclize and radiolabel octreotide, in other embodiments, DOTAGALP1 may be used to cyclize other peptides containing multiple cysteines, of various length and amino acid composition. In particular, DOTAGALP1 can be applied to cyclic peptides containing a disulphide bridge for tandem peptide cyclization/labeling with a metal chelator for subsequent radiolabeling.

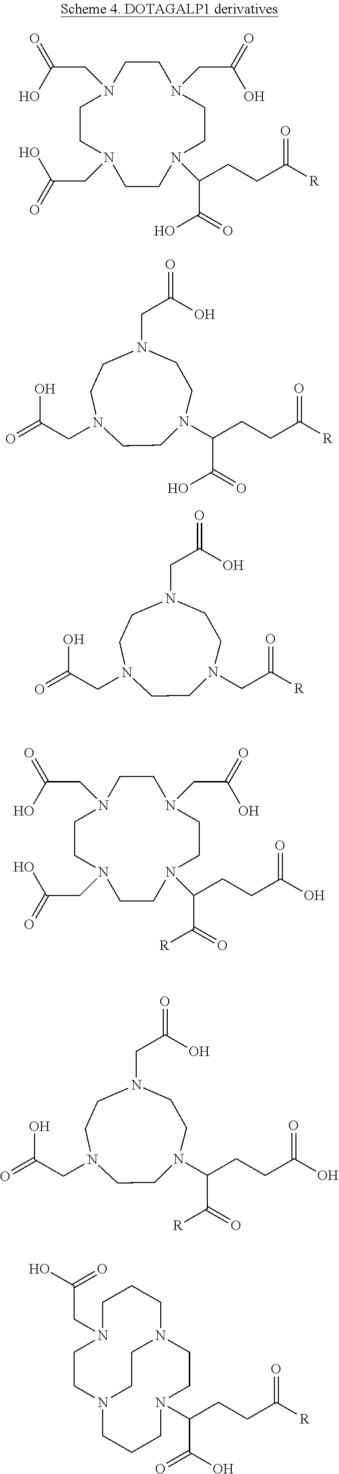

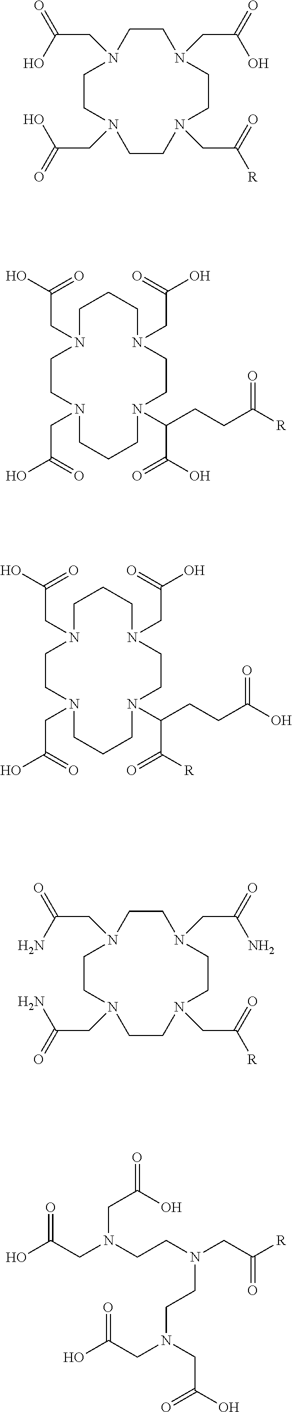

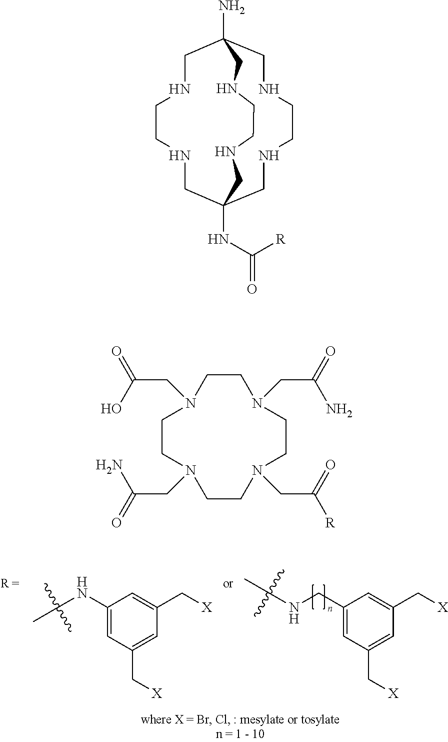

### Example 2

### Synthesis of SiFALP

In addition to DOTAGALP1, a different class of bifunctional linchpins has been developed. These linchpins involve the use of a dibromomaleimide as the cyclization motif. While chelators continue to be explored, a SiFA-motif (for silicon-acceptor chemistry) can also be incorporated into the structure to access 18F-labelled compounds (SiFALP) easily. These maleimide derivatives can be easily synthesized from the reaction shown in Scheme 5.

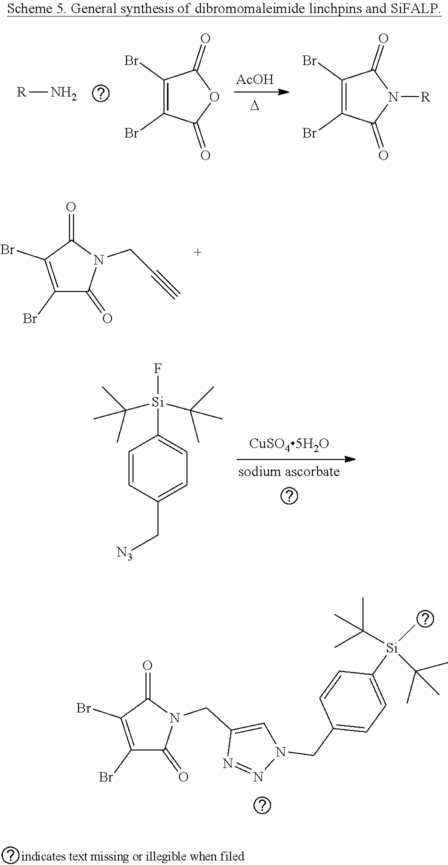

SiFALP was also used to cyclize octreotide in a model reaction (Scheme 6). The corresponding product Octreo-SiFA was subjected to the same assay against SSTR1-5 and showed good inhibitory activity against SSTR2 (Table 2).

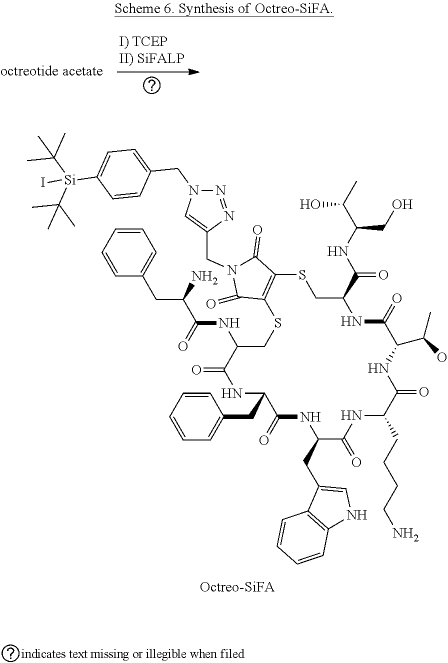

TABLE 2
IC₅₀ values (nM) of SiFALP functionalized octreotide
against somatostatin receptors (SSTR) in [¹²⁵I]SST14
binding competition

| Compound | SSTR1 | SSTR2 | SSTR3 | SSTR4 | SSTR5 |
|---|---|---|---|---|---|
| SST28 ref. | 1.32 | 0.32 | 0.35 | 2.00 | 0.10 |
| Octreo-SiFA | 5090 | 21.4 | 211 | 6809 | 100 |

Representative examples are shown below:

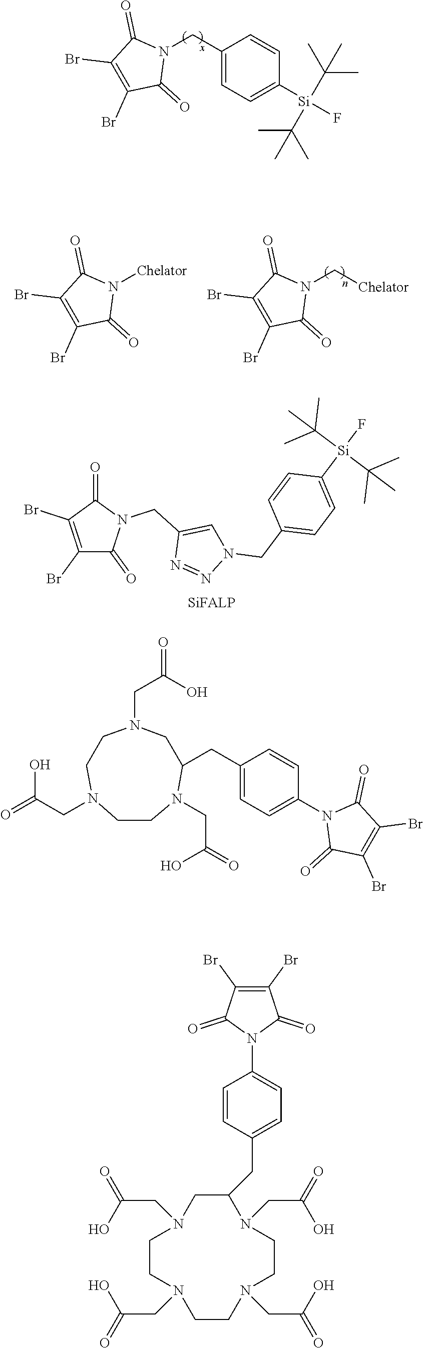

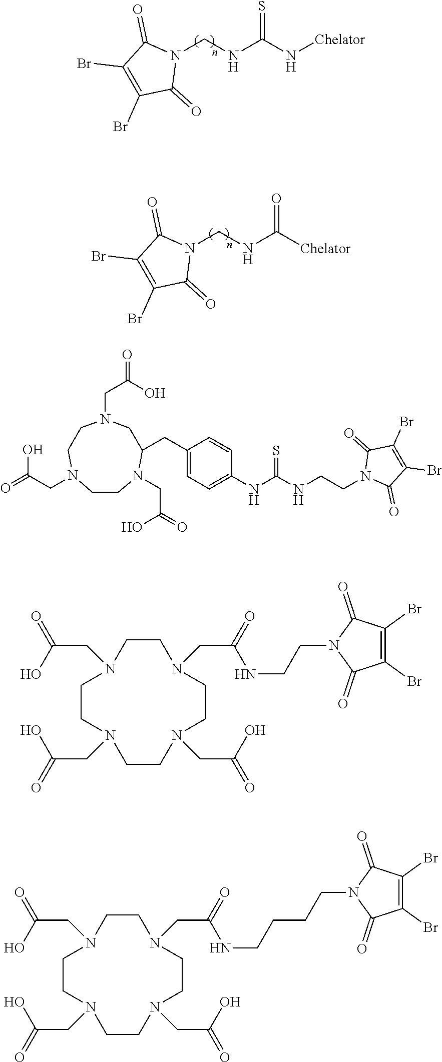

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A compound of Formula Y-L-R,
wherein R is 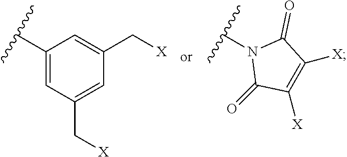
and X is a halogen, mesylate, or tosylate;
and L is an optional linker; 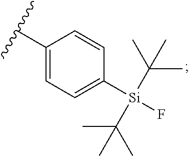 [SMILES: *c1ccc([Si](F)(C(C)(C)C)C(C)(C)C)cc1]
and Y is a radiometal chelator or
and wherein the compound is optionally further labeled with a radiometal.

2. The compound of claim 1, wherein the halogen is Cl, Br, or I.

3. The compound of claim 1, wherein the mesylate is methanesulfonyl.

4. The compound of claim 1, wherein the tosylate is p-methylphenylsulfonyl.

5. The compound of any one of claims 1 to 4, wherein the radiometal chelator is selected from one of:

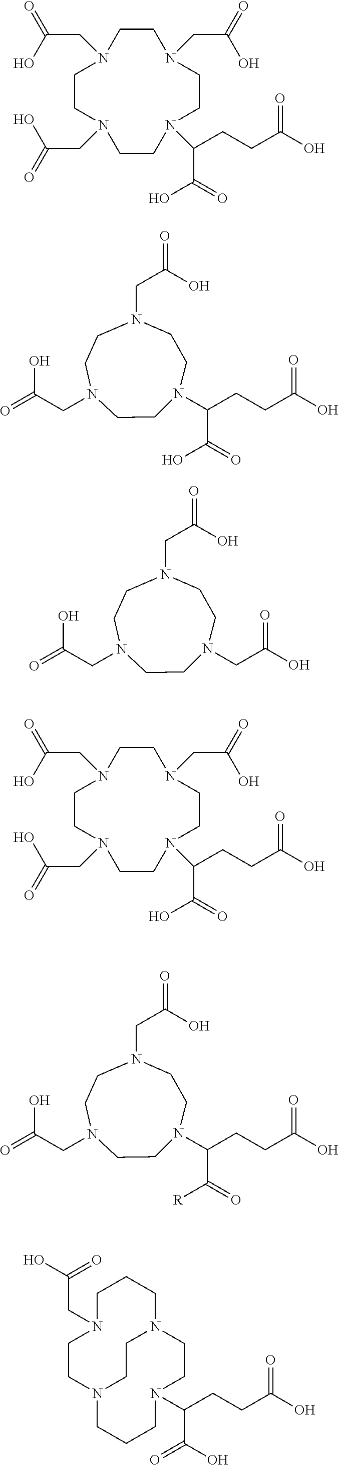

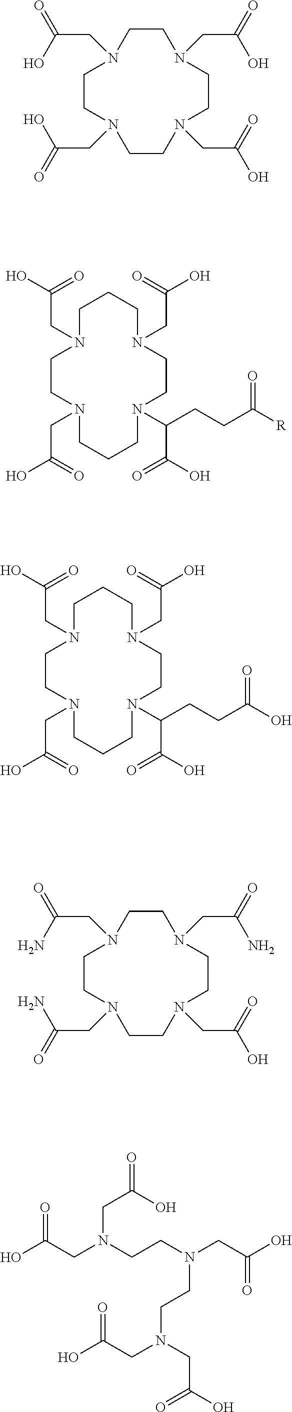

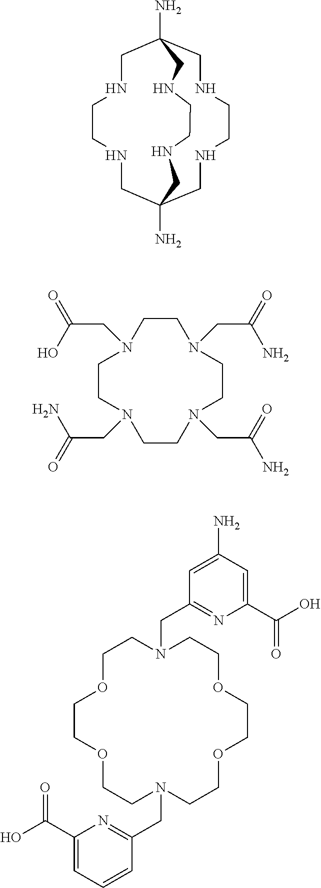

6. The compound of any one of claims 1 to 5, wherein the radiometal is I¹²⁵, ⁶⁷Cu, ⁶⁸Ga, or ¹³⁵La.
